(19) United States
(12) Patent Application Publication
(10) Pub. No.: US 2013/0085571 A1
(43) Pub. Date: Apr. 4, 2013

(54) COMPOSITION AND METHOD OF PREPARATION OF POLYSACCHARIDE GEL-BASED ARTIFICIAL, BIODEGRADABLE SKIN SCAFFOLDS

(71) Applicants: Anja Mueller, Mount Pleasant, MI (US); Robert D. Kross, Bellmore, NY (US)

(72) Inventors: Anja Mueller, Mount Pleasant, MI (US); Robert D. Kross, Bellmore, NY (US)

(21) Appl. No.: 13/695,165

(22) PCT Filed: May 14, 2010
(86) PCT No.: PCT/US10/35034

§ 371 Date: Oct. 29, 2012

Related U.S. Application Data
(60) Provisional application No. 61/178,246, filed on May 14, 2009.

(51) Int. Cl. A61F 2/10

(52) U.S. Cl. CPC A61F 2/105
USPC 623/15.12; 600/36

(57) ABSTRACT

An artificial, biodegradable skin scaffold includes a biodegradable polysaccharide hydrogel composition having a Young's modulus of at least about 10 MPa and, preferably, includes an additional biodegradable polysaccharide hydrogel composition with the two biodegradable polysaccharide hydrogel compositions contacting one another and the additional biodegradable polysaccharide hydrogel composition preferably having a Young's modulus of less than about 10 MPa.

### CROSS-REFERENCE TO RELATED APPLICATIONS

This application claims priority under 35 U.S.C. §119(e) to U.S. Provisional Patent Application No. 61/178,246 filed on May 14, 2009. The entire contents of this application are hereby incorporated by reference in its entirety.

### BACKGROUND

The skin is the largest organ in the body and serves many functions such as protection against infection or injury, immune surveillance, perception of touch or pain, and regulation of temperature and water loss. The skin is divided into two layers: the epidermis, which is composed mainly of keratinocytes that form the thin and tough protective layer and contains hair follicles and glands, and the thicker and more porous dermis, which contains many different cell types, including collagen-producing fibroblasts, blood vessel-forming endothelial cells, motor and sensory neurons, and immunoregulatory cells.

When skin is superficially damaged, the wound is rapidly repaired. However, when the injury destroys both epidermis and dermis, patients often die from infection or loss of plasma. If patients survive with the help of skin grafts, the skin regenerates but often heals with the formation of abundant scar tissue and without the regeneration of some cell types, including hair follicles and sebaceous glands.

Artificial skin often contains animal material, such as collagen, which may contain infectious material. Biodegradable hydrogels are generally not physically strong enough to mimic the tough, thin epidermis. A polymer system often used in skin scaffold synthesis is a polyester based on lactic acid (“PLA”) and glycolic acid (“PGA”). However, these polyesters have been shown to degrade to their acidic components, resulting in a high local acidity that can destroy proteins.

### SUMMARY

The present application provides improved methods and materials for fabricating artificial skin. The present application provides artificial, biodegradable skin scaffolds including biodegradable polysaccharide hydrogel composition layers.

Embodiments in accordance with the present invention provide an artificial, biodegradable skin scaffold including a first biodegradable polysaccharide hydrogel composition, having a Young's modulus of at least about 10 MPa. Some embodiments may further include a second biodegradable polysaccharide hydrogel composition. In some embodiments, the second biodegradable polysaccharide hydrogel composition may contact the first biodegradable polysaccharide hydrogel composition. In some embodiments, the second biodegradable polysaccharide hydrogel composition may have a Young's modulus of less than about 10 MPa. Other embodiments may further include a third biodegradable polysaccharide hydrogel composition, where the first biodegradable polysaccharide hydrogel composition is layered between the second and third biodegradable polysaccharide hydrogel compositions.

In some embodiments, the first biodegradable polysaccharide hydrogel composition can include a polysaccharide selected from carrageenan, xanthan gum, locust bean gum, konjac gum, starch, methyl cellulose, carboxymethyl cellulose, ethyl cellulose, partially- or fully-deacetylated gellan, carob gum, agar, TICAGEL®121-AGF, poly(glucuronic acid), poly(galacturonic acid), or a combination thereof. In some embodiments, the polysaccharide in the polysaccharide hydrogel composition is plant-derived.

In some embodiments, the first biodegradable polysaccharide hydrogel composition includes a glycol ether, a pyrrolidone, a carboxymethyl cellulose, an organic acid, a monosaccharide, a disaccharide, an antibacterial agent, a surfactant, or a combination thereof.

In some embodiments, the first biodegradable polysaccharide hydrogel composition comprises less than about 4.2% konjac gum. In some embodiments, the first biodegradable polysaccharide hydrogel composition comprises less than about 2.8% xanthan gum. In some embodiments, the first biodegradable polysaccharide hydrogel composition comprises less than about 2% κ-carrageenan. In some embodiments, the first biodegradable polysaccharide hydrogel composition comprises less than about 4% ι-carrageenan. In some embodiments, the first biodegradable polysaccharide hydrogel composition comprises at least three polysaccharides selected from the group consisting of konjac gum, xanthan gum, κ-carrageenan, and ι-carrageenan.

In some embodiments, the first biodegradable polysaccharide hydrogel composition comprises a biological signal molecule. In some embodiments, the biological signal molecule is contained within a particle of a polysaccharide hydrogel composition. In some embodiments, the biological signal molecule comprises a peptide, a protein, or a pharmaceutical. In some embodiments, the first biodegradable polysaccharide hydrogel composition comprises a wound-healing peptide.

In some embodiments, the artificial, biodegradable skin scaffold further includes skin cells. In some embodiments, the skin cells are autologous. In some embodiments, the skin cells are selected from the group consisting of fibroblasts, keratinocytes, or a combination thereof.

Another embodiment provides an artificial, biodegradable skin scaffold comprising a biodegradable polysaccharide hydrogel composition, where the biodegradable polysaccharide hydrogel composition comprises a biological signal molecule. In some embodiments, the biodegradable polysaccharide hydrogel composition has a Young's modulus of less than about 10 MPa. In some embodiments, the biological signal molecule is contained within a particle of a polysaccharide hydrogel composition. In some embodiments, the biological signal molecule comprises a peptide, a protein, or a pharmaceutical.

Another embodiment provides a method of preparing an artificial, biodegradable skin scaffold comprising: preparing a first biodegradable polysaccharide hydrogel composition; and drying the first biodegradable polysaccharide hydrogel composition, where the first biodegradable polysaccharide hydrogel composition has a Young's modulus of at least about 10 MPa after drying. In some embodiments, the drying step removes about 70 wt. % to about 80 wt. % of water from the first biodegradable polysaccharide hydrogel composition.

In some embodiments, the method further comprises preparing a second biodegradable polysaccharide hydrogel composition and drying the second biodegradable polysaccharide hydrogel composition, where the second biodegradable polysaccharide hydrogel composition has a lower Young's modulus than the first biodegradable polysaccharide gel. In some embodiments, the method further includes seeding the second biodegradable polysaccharide hydrogel composition with fibroblasts. In some embodiments, the Young's modulus of the second biodegradable polysaccharide hydrogel composition is less than 10 MPa. In some embodiments, at least one of the first and second biodegradable hydrogels comprise poly(glucuronic acid), poly(galacturonic acid), or a combination thereof. In some embodiments, the method further comprises crosslinking the polysaccharide with a polyalkylkene glycol.

### BRIEF DESCRIPTION OF THE FIGURE

FIG. 1 shows a ¹H NMR of poly(glucuronic acid) crosslinked with different length polyethylene glycols.

### DETAILED DESCRIPTION

The invention provides an artificial, biodegradable skin scaffold based on a biocompatible polysaccharide. The artificial, biodegradable skin scaffold can be used to treat or heal wounds in which the skin is damaged or missing, such as occurs with third degree burns.

As used herein, the term “biodegradable” refers to the ability of a material to be broken down into less complex intermediates or end products, e.g., means a polymer that is susceptible to degradation into shorter chain carbohydrate fragments through hydrolysis, through action by bacterial or human enzymes, due to topical and/or environmental factors, or other degradative mechanisms.

As used herein, the term “hydrogel” refers to a gel (e.g., a network of polymer chains) in which water is the dispersion medium, e.g., a polymeric material that exhibits the ability to swell in water and to retain a significant portion of water within its structure without dissolving.

As used herein, the term “derived from animals” means that the components are of animal origin and includes those that are modified or altered, but does not include synthetic components that may mimic an animal component.

Before any embodiments of the present application are explained in detail, it is to be understood that the invention is not limited in its application to the details of construction and the arrangement of components set forth in the following description or illustrated in the following drawings. The invention is capable of other embodiments and of being practiced or of being carried out in various ways.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range or a beneficial effect range is stated as 1% to 50%, it is intended that values such as 2% to 40%, 10% to 30%, or 1% to 3%, etc. are expressly enumerated in this specification. These are only examples of what is specifically intended.

Embodiments of the invention provide an artificial, biodegradable skin scaffold is composed of biodegradable polysaccharide hydrogel compositions having a range of mechanical and degradation properties sufficient to fulfill the different functions of artificial skin. These include, for example, protection, scaffolding for different cell types, biodegradation or bioresorbability, and ability to release the signals for cell growth and wound healing.

In one embodiment, the invention provides an artificial, biodegradable skin scaffold containing a biodegradable polysaccharide hydrogel composition layer of a mixture of polysaccharides which mimics the tough dermis of the skin. Commonly, the hydrogel may have a Young's modulus of at least about 10 MPa.

In another embodiment, the invention provides an artificial, biodegradable skin scaffold containing a biodegradable polysaccharide hydrogel composition layer of a mixture of polysaccharides and having a Young's modulus of less than about 10 MPa.

In another embodiment, the invention provides an artificial, biodegradable skin scaffold composed of a multilayer biodegradable polysaccharide hydrogel composition system. The scaffold includes a first biodegradable polysaccharide hydrogel composition layer containing a polysaccharide mixture and a second biodegradable polysaccharide hydrogel composition layer containing a mixture of polysaccharides and which contacts the lower layer. The polysaccharides in the second layer may, for example, be related to (i.e., be the same or substantially similar to) the polysaccharides in the first layer. For example, the first and second layers may contain the same or similar types or combinations of polysaccharides, the same or similar ratios of polysaccharides (relative to each other by weight), and/or the same or similar amounts (by weight) of polysaccharides. In some embodiments, the first layer may have a higher Young's modulus than the second layer. In some embodiments, the Young's modulus of the first layer may be at least about 10 mPa. In some embodiments, the Young's modulus of the second layer may be less than about 10 mPa.

When applied to the skin, the second layer contacts the skin or wounded area (and may be referred to as the lower layer), and lies between the skin or wound and the first layer (which may be referred to as the upper layer). The multilayer hydrogel system may also include a third layer, which overlays the first layer (which may be collectively referred to as the upper layers).

In one embodiment, the second or lower layer of hydrogel contains a polysaccharide mixture and has a greater surface area and larger pore size than the first or other upper layers. In some embodiments, the second layer pore diameters may be from about 0.1 μm to about 10 μm. In some embodiments, the second layer may have a Young's modulus of less than about 10 mPa, less than about 5 MPa, or less than about 1 MPa. In some embodiments, the second layer may be more cohesive than the first or other upper layers. The first or other upper layers of hydrogel contain a polysaccharide mixture and are suitably denser and/or stronger than the second or lower layer.

The artificial, biodegradable skin scaffold may also include an optional cover layer containing one or more polysaccharides. Suitably the cover layer is composed of a biodegradable polysaccharide hydrogel composition. The optional cover layer forms the top layer overlaying the first and second layers. In a multilayer hydrogel system, for example, the optional cover layer suitably contacts a first hydrogel layer, the first hydrogel layer contacts a second hydrogel layer and the second hydrogel layer contacts the skin or wound area.

In one embodiment, the invention further provides artificial, biodegradable skin scaffolds containing biodegradable polysaccharide hydrogel compositions into which biological signal molecules have been incorporated. Biological signal molecules include, without limitation, peptides, proteins, pharmaceuticals. For example, keratinocyte growth factor (“KGF”) which stimulates keratinocyte proliferation and migration and/or basic fibroblast growth factor (“bFGF”) which promotes growth of endothelial cells and angiogenesis may be used. Members of the interleukin-1 family of cytokines that are master regulators of inflammation and down regulation of the inflammatory response may also be used. One or more of a combination of these molecules will allow wound healing to occur with minimal scarring.

In some embodiments, the biological signal molecule may be free within the biodegradable polysaccharide hydrogel composition. For example, the biological signal molecules can be either diffused through the lower and/or upper layers. In one embodiment, the biological signal molecule is contained within a polysaccharide particle. The polysaccharide particle or hydrogel suitably facilitates delivery of the molecule to its intended target. For example, the composition of hydrogel layers may be tailored to provide controlled release of the biological signal molecules, so that they are released in amounts and at times to promote wound healing, as known in the art. (For methods of preparing biological signal molecules contained within a polysaccharide particle and providing controlled release of biological signal molecules, see, e.g., Wang, et al., “Microencapsulation Using Natural Polysaccharides for Drug Delivery and Cell Implantation,” Journal of Materials Chemistry 2006, 16, 3252-3267; and Peattie et al., “Stimulation of In Vivo Angiogenesis by Cytokine-Loaded Hyaluronic Acid Hydrogel Implants,” Biomaterials 2004, 25, 2789-2798.) Polysaccharide mixtures may be prepared to have adjusted degradation kinetics to accommodate the biological need of the specific signal at a specific time of the healing process. In other embodiments, the biological signal molecule may be bound, either physically or chemically, to the hydrogel.

Polysaccharides useful in the present application may be obtained or derived from plants and/or synthesized by bacteria and/or synthesized in vitro using enzymes or other techniques such as organic synthesis. Polysaccharides obtained or derived from plants may be used in unmodified form, or may be chemically-modified (for example, carboxymethyl cellulose). Suitably, the polysaccharide is non-animal, i.e., is not obtained or derived from animals, and excludes mammalian-derived, avian-derived, or piscean-derived polysaccharides. The polysaccharide may be used in hydrogels that when gelled are free of, or of, animal or animal-derived components. Suitably, each of the components which contribute to the gelling of the hydrogel are not obtained or derived from animals, including non-human animals.

The use of in vitro techniques permits synthesis of the polysaccharide in a controlled manner. Suitably synthesis can be carried out with an aqueous solvent and an enzyme catalyst. Enzymatic synthesis facilitates the incorporation of biological signal molecules, such as signal proteins, to be incorporated into the polymerization and/or crosslinking reaction, allowing for a homogeneous distribution of bioactive proteins in the material. Suitable enzymes include glycosyltransferases and glycosidases, and horseradish peroxidase (“HRP”). HRP is a redox enzyme that can polymerize various phenols, as well as other aromatic and vinyl monomers, in the presence of hydrogen peroxide as the oxidant. HRP may be used to polymerize glucuronic acid and related sugars. Poly(glucuronic acid) with a molecular weight of up to 60,000 is suitably synthesized in a repeatable and controlled manner. Lipase may be used for crosslinking of poly(glucuronic acid) with poly(ethylene glycol) (PEG). Poly(glucuronic acid) can be crosslinked with PEG of different chain lengths in either water alone or in aqueous buffer as the solvent. Polysaccharides may be also synthesized by, for example, Xanthomonas campestris, or by bacteria genetically engineered to produce a polysaccharide.

Polysaccharides suitable for use in biodegradable polysaccharide hydrogel compositions and artificial, biodegradable skin scaffolds of the invention are those that have a particular ability to interact with each other to form 3-dimensional, stable structures. These include, without limitation, one or more of konjac gum, konjac flour, xanthan gum, carrageenan (such as κ-carrageenan, ι-carrageenan), locust bean gum, and combinations thereof. Other polysaccharides which contribute beneficial qualities to these 3-dimensional structures include, without limitation, carboxymethyl cellulose or its alkaline salts, starch, methyl cellulose, ethyl cellulose, partially- or fully-deacetylated gellan, carob gum, agar, TICAGEL®121-AGE, or combinations thereof. Suitably, the artificial, biodegradable skin scaffold and hydrogel layers contain a mixture of one or more polysaccharides extracted from plants and one or more polysaccharides synthesized in vitro. The polysaccharides extracted from plants may be separated or purified into fractions of a particular molecular weight. Thus, many hydrogels of different mechanical strengths and degradation rates can be made by mixing and matching different polysaccharides having controlled properties.

A suitable biocompatible hydrogel comprises one or more of the following components: N-methyl pyrrolidone (optional), konjac gum, κ-carrageenan gum, ι-carrageenan gum, carboxymethyl cellulose (optional), a potassium salt, a calcium salt glucose or a related mono- or di-saccharide, malic acid or a related organic acid, glycerine or related glycols, and/or preservatives.

Suitable preservatives are known to those skilled in the art, and include, but are not limited to, sodium benzoate and COSMOCIL®CQ.

Suitable biodegradable polysaccharide hydrogel compositions may be fabricated from components included in Table 1, in the concentration ranges provided:

TABLE 1
Hydrogel Components

| COMPONENT | PERCENT BY WEIGHT |
|---|---|
| N-Methyl Pyrrolidone | 0-30.0 |
| Konjac gum (e.g. TIC Konjac- | 0-4.2 |
| High Viscosity Flour) | |
| Xanthan gum (e.g. Ticaxan Rapid | 0-2.8 |
| Powder) | |
| κ-Carrageenan gum (e.g. TIC | 0-2.0 |
| gum 710H100) | |
| ι-Carrageenan, i- gum (e.g. | 0-4.0 |
| Colloid 881M) | |
| Potassium chloride | 0-2.0 |
| Calcium Lactate | 0-4.0 |
| Sodium benzoate | 0-1.0 |
| COSMOCIL ® CQ | 0-1.0 |
| Glucose | 0-22.5 |
| Malic acid | 0-0.25 |
| Water, DW/DI/RO | 10.0-99.0 |

Optional and/or alternative ingredients in the biodegradable polysaccharide hydrogel compositions include solvents of the CARBITOL™ series (commercially available from Dow Chemical Company), including, for example, diethyleneglycol monoethyl ether, and analogs such as the propyl and butyl ethers, and related dipropyleneglycol monoethyl ether mono-hydroxy compounds, where the other hydroxy function has been etherified; organic acid acidifiers; mono- and disaccharides; antibacterial agents; and surfactants.

The biodegradable polysaccharide hydrogel composition suitably contains glycols, such as polyethylene glycol, and others as taught in U.S. Pat. No. 6,664,301, which is incorporated by reference herein in its entirety. In some embodiments, the hydrogel does not contain glycols. Hydrogels can be generally formed by pre-dispersing one or more of the polysaccharides in a small amount of a water-compatible medium, if applicable, and then introducing the dispersion into an aqueous medium which is then heated and stirred to a point of dissolution and uniformity. In various embodiments, the dispersion is heated at a temperature of about 40° C. to about 95° C. In various embodiments, the dispersion is heated from a time of from about 0.5 minutes to about 60 minutes. The mixture is then cooled to a point where a gel is formed. In various embodiments, this corresponds to a temperature of from about 4° C. to about 40° C. Glycols and other optional ingredients can generally be added to the polysaccharide before or during heating in the aqueous medium. These additional optional ingredients can be added to impart any of a range of desired properties, including, but not limited to, gel strength, clarity, biocompatibility, degradation kinetics, polysaccharide structure, protein structure protection, surface area, cell adhesion, pore size and/or antibacterial properties.

Once a biodegradable polysaccharide hydrogel composition is formed, it can be dried to remove a portion of the aqueous phase. This drying process can be carried out either on the individual layers prior to their combination, or to the complete carbohydrate scaffold. The drying process can be carried out over a variety of different time, temperature, and relative humidity conditions. For example, the hydrogel may be dried for a period of from about 1 to about 30 days at a temperature of from about 4° C. to about 25° C. In some embodiments, drying can remove at least about 10%, at least about 15%, or at least about 20% of the aqueous phase from the hydrogel. In some embodiments, drying can remove about 10% to about 20% of the aqueous phase from the hydrogel. In some embodiments, drying can remove at least about 70%, at least about 80%, or at least about 90% of the aqueous phase from the hydrogel. In some embodiments, drying can remove about 70% to about 80% of the aqueous phase from the hydrogel. The drying operation suitably increases the strength of the hydrogel while also modifying the degradation kinetics of the final product.

The biodegradable polysaccharide hydrogel compositions are formed from polysaccharides that are biocompatible and/or biodegradable. Degradation of the polysaccharides and/or hydrogels may depend on temperature, pH, molecular weight, crystallinity, surface-to-volume ratio, pore size, and enzyme concentration.

The biodegradable polysaccharide hydrogel compositions may be sterilized prior to or after assembly into the artificial, biodegradable skin scaffold. Suitably, sterilization may be carried out using high-intensity pulsed Xenon laser beams or electron beams or through the use of chemical sterilants.

The skin scaffold is formed by assembling one or more of the biodegradable polysaccharide hydrogel compositions into layers. Suitably the skin scaffold contains at least 1, and up to 3 layers. Each of these layers may be the same or different in carbohydrate composition. When two or more layers are present, the lower layer which contacts the wound area or skin is suitably a softer and less dense hydrogel than the hydrogels present in one or more of the upper layers. Suitably, one or all of the hydrogel layers are biocompatible and/or biodegradable. In one embodiment, it is envisaged that assembly of the layers of the skin scaffold may be performed just prior to application of the scaffold to the wound area or skin. Alternatively, the skin scaffold may be assembled prior to use.

In one embodiment, cells, such as fibroblasts and/or keratinocytes, are provided in the artificial, biodegradable skin scaffold. In some embodiments, the skin scaffold is assembled and seeded with cells prior to use. The cells may be allowed to multiply in the scaffold prior to application to the subject. The cells are suitably autologous. For example, fibroblasts, such as those obtained or derived from the subject having the wound to be treated, can be seeded on or in the lower layer contacting the skin or wound. In a multi-layer scaffold, the subject's own keratinocytes may also be seeded on or in the overlying upper layer. Methods for seeding the biodegradable polysaccharide hydrogel composition layers include, without limitation, seeding cells on top of the preformed layer, allowing cells to diffuse from a liquid medium into the preformed layer, or combining cells with the still-liquid mixture prior to gel formation. The biodegradable polysaccharide hydrogel compositions may be seeded prior to assembly into a skin scaffold, or may be seeded after assembly.

Biodegradable polysaccharide hydrogel compositions may also be formed having three-dimensional patterns that facilitate growth of the cells. For example, the hydrogel may contain ridges or channels to influence the differentiation of cells such as keratinocytes. Channels may permit stratification of cells such as keratinocytes.

A porous and permeable network in the biodegradable polysaccharide hydrogel composition and/or scaffold can be created by several methods. Artificial, biodegradable skin scaffolds can be formed, for example, by layering thin films of polysaccharide materials containing a bioactive protein. Each layer may be initially crosslinked in situ under sterile conditions. It is also possible to comminute the bioactive releasing materials into small particles, and crosslink the scaffold material around the bioactive materials. Since polysaccharides often have protective properties towards bioactive materials, the gel may be formed at a higher temperature than where the bioactive materials might otherwise survive. Alternatively, the bioactive materials may be dispersed in the cooling mixture before significant gelling begins.

The biodegradable polysaccharide hydrogel composition which may form one or more upper layers, is suitably more dense and/or stronger than the lower layer that contacts the skin or wound area, while still being biocompatible and/or bioresorbable. The higher relative density is a function of the specific mixture employed and the initial and final water content of that layer. The upper layer is also biocompatible and/or biodegradable.

### Measurement of the Young's Modulus of a Hydrogel

Mechanical analysis of hydrogels of the present application can be conducted, for example, with a TAQ800 Dynamic Mechanical Analyzer (“DMA”) via a strain ramp, from 0.01%-0.4%. The Young's modulus may be calculated from the slope of the linear region of a stress vs. strain curve using TA universal analysis software.

### Method of Describing the Degree of Intermolecular Hydrogen Bonding in a Hydrogel

Hydrogels of the present application can be desiccated, made into KCl pellets, and their infrared spectra recorded in an infrared spectrometer, such as a Nicolet Magna IR 560 spectrometer, over the frequency range including from about 4000 cm⁻¹ to 2000 cm⁻¹. The —OH peak, present on the spectra in the range from about 3000 cm⁻¹ to about 3500cm^{31 1}, can be used to determine the relative H-bonding strength of the gels' intermolecular hydrogen bonds.

The calculation begins with the vertical division of the —OH peak on the IR recording into about 50 cm⁻¹ increments, starting at either side of the —OH peak, and assigning sequential numbers (e.g., 1 to10), to each 50 cm⁻¹ increment. The height of each of these increments from the baseline of the spectral peak to the peak maximum can then be measured in millimeters and added together. This sum is divided by 100 to provide the division factor. Beginning at the higher frequency end of the peak, each increment within the peak is then multiplied by its corresponding sequential number (i.e., 1, 2, 3, etc.) and divided by the division factor, resulting in a weighted average. All of these calculated numbers are then totaled, and that number is reflective of the degree of H-bonding strength, i.e., a hydrogen bonding value (“HBV”). Higher HBVs indicate less hydrogen bonding. If the sample contains NH bonds as well, only samples with the same amount of OH and NH peaks can be compared with each other.

### EXAMPLES

### Experimental Materials Used in Examples

HPLC grade water and 30% hydrogen peroxide were purchased from EMD Chemicals; potassium phosphate monobasic and dibasic from Fisher Scientific; Triton X-100, Bovine Serum Albumin (BSA), 2,2′-Azino-bis(3-ethylbenzthiazonoline-6-sulfonic acid) (ABTS), D-(+)-glucuronic acid, horseradish peroxidase (HRP, Type VI-A, Sigma P-6782, 1,280 units/mg) Sigma.

### Instrumentation Used in Examples.

UV spectra were obtained on an Agilent 8453 UV/V is Spectrometer utilizing Agilent ChemStation software (A.08.03). Gel permeation chromatography was conducted on a Waters Breeze system consisting of a Waters 1515 isocratic pump, a Waters 717+ autosampler, and a Waters 2414 refractive index detector with the column heater module with Breeze hardware and software and Fluka polyethylene glycol (“PEG”) standards. Two TsoHaas TSK-Gel 7.5 mm×30 cm columns were connected in series and maintained at 30° C. with deionized ultra-filtered (DIUF) water as solvent at 1.0 ml/min. For IR spectroscopy, the gels were desiccated, made into KCl pellets, and run in a Nicolet Magna IR 560 spectrometer. Differential scanning calorimetry (“DSC”) was carried out at a heating rate of 10° C./min using a TA Instruments Inc. model 2100 equipped with a model 2910 DSC cell at a constant purge of dry nitrogen at 50 ml/min. A TA Instruments model 2950 thermal gravimetric analysis (“TGA”) unit was interfaced with the Thermal Analyst model 2100 control unit and swept with nitrogen at 50 ml/min for TGA measurements; 5-10 mg of sample in a platinum sample pan was heated 10° C./min. A TAQ800 Dynamic Mechanical Analyzer (“DMA”) was used for the mechanical analysis of the gels via a strain ramp from 0.01%-0.4%. The Young's modulus was calculated from the slope of the linear region of a stress vs. strain curve using TA universal analysis software.

### Example 1

### Hydrogen Preparation

A hydrogel was prepared from the components listed in Table 2, using the process detailed below.

TABLE 2
Hydrogel Components

| COMPONENT | # | % | COMMENTS |
|---|---|---|---|
| N-Methyl | 1 | 6.00 | Non-toxic, water-soluble |
| Pyrrolidone | | | dispersant for gum powders |
| Konjac gum | 2 | 0.84 | High Viscosity Flour |
| Xanthan gum | 3 | 0.56 | Rapidly Dissolving Powder |
| κ-Carrageenan gum | 4 | 0.40 | Rapidly Dissolving Powder |
| ι-Carrageenan gum | 5 | 0.80 | iota-carrageenan and maltodextrin mix |
| Potassium chloride | 6 | 0.40 | Optimizes function of κ-Carrageenan |
| Calcium Lactate | 7 | 0.80 | Optimizes function of ι-Carrageenan |
| Sodium benzoate | 8 | 0.10 | Preservative |
| COSMOCIL ® CQ | 9 | 0.10 | Preservative |
| Glucose | 10 | 4.50 | facilitates intermolecular bonding |
| Malic acid | 11 | 0.05 | H⁺ ion strengthens hydrogel structure |
| Water, DW/DI/RO | 12 | q.s. | Solvent [85.45%] |

Method of Preparation (Numbers Refer to Components Listed at the corresponding Lines in Table 3 Above)

a) Component 1 was weighed in a suitable beaker, and then components 2, 3, 4, and 5 were added. The powders were stirred with a flat spatula to wet the powders then, with the spatula in beaker, the balance was re-zeroed and components 6, 7, 8, & 9 were added. The components were mixed until uniform throughout.

b) Components 10 and 11 were mixed separately, and set aside.

c) The required amount of water was added to mixture from a) and once disperse and/or dissolved, the mixture from b) was added and stirred to dissolve.

d) The mixture was stirred until it began to thicken. It was weighed, and then heated (covered) with intermittent stirring to about. 85° C. The mixture was stirred again, weighed and additional water was added to regain lost weight. The mixture was reheated to 85° C. (entrapped air bubbles may be removed by brief placement in a vacuum chamber, prior to cooling), stirred and poured into an appropriate mold. The hydrogel was covered with a suitable plastic sheet (e.g., PE, Saran) and allowed to cool and gel.

e) The hydrogel may be removed from the mold (optional) and stored in a sealable container if drying of the hydrogel is not desired. If drying of the hydrogel is desired, the gel can be dried in air at an appropriate temperature (e.g., 25° C., 4 ° C., etc.) over a specified number of hours or days (e.g., 12 hours, 1 day, 3 days, etc.).

General Considerations for Example 1 (Numbers Refer to Components Listed at the Corresponding Lines in Table 2 Above)

(1) It is generally helpful to pre-disperse the gum components in a non-aqueous hydrophilic dispersant, so that the subsequently-introduced water can slowly replace the absorbed dispersant. The dispersant should be compatible with skin tissue.

(2) Konjac gum, as a natural material, is subject to natural variations in degree of polymerization and, accordingly, the viscosity of the thickened aqueous solution it creates. Konjac gums commercially available from TIC Gums Inc., for example, are suitable and fall in a defined viscosity range. Konjac interacts synergistically with a number of gums, specifically κ-carrageenan, xanthan gum, to form self-supporting three-dimensional hydrogels.

(3) Suitable xanthan includes that isolated commercially from fermentation cultures of Xanthomonas campestris. Xanthan does not form a gel when used alone, but it can form a 3-dimensional cohesive gel in a broad pH range when used in certain combinations with konjac gum.

(4) Carrageenan is a collective term for polysaccharides prepared by alkaline extraction (and modification) from red seaweed. Different red seaweeds produce different carrageenans. They consist of alternating 3-linked-β-D-galactopyranose and 4-linked -α-D-galactopyranose units. κ-Carrageenan (carrageenose 4′-sulfate (G4S-DA)) differs from ι-carrageenan (carrageenose 2,4′-sulfate (G4S-DA2S)) in its in its steric conformation, which allows for a different and stronger type of intermolecular bonding for the κ-carrageenan-konjac combination. Suitably the latter may include K⁺ ion to enhance functionality, whereas the ι-carrageenan suitably forms tight bonds in the presence of Ca²⁺ ions. As indicated, the konjac/κ-carrageenan gum combination adds firmness to the hydrogel structure involving konjac/xanthan.

(5) The ι-carrageenan gum powder used here is a mixture of the basic gum and a maltodextrin diluent, which is presently commercially available TIC Gums Inc., for example, in variable amounts to standardize the product to a standard viscosity range.

(8, 9) The benzoate and COSMOCIL® (polyaminopropyl biguanide) preservatives are well tolerated in topical products for cosmetic application. These components are optional, and appropriate alternatives may or may not be employed.

(10) Glucose may be replaced in whole, or in part, by related sugars to achieve certain desired physical effects.

### Example 2

### Synthesis of a Polysaccharide In Vitro

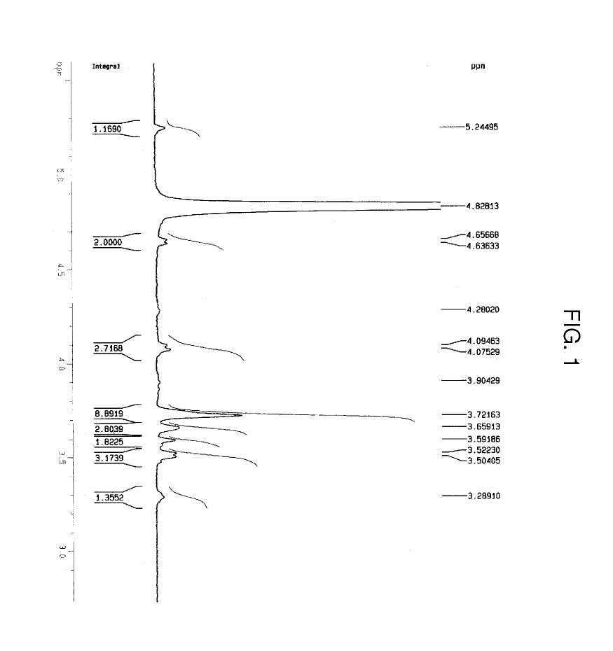

Poly(glucuronic acid) was crosslinked with different length polyethylene glycols (“PEG”). The mixture was reacted with lipase at 37° C. for 5 days. The new peak at 3.65 ppm in the ¹H NMR (FIG. 1) demonstrates the formation of the ester bond of the crosslink (represented in Scheme 1 below). Integration of this and the neighboring peak show that 80%-95% of the PEG had both of its hydroxyl end groups reacted with poly(glucuronic acid), indicating that this is an efficient crosslinking method. The material changed from a viscous liquid to a solid in all cases upon crosslinking.

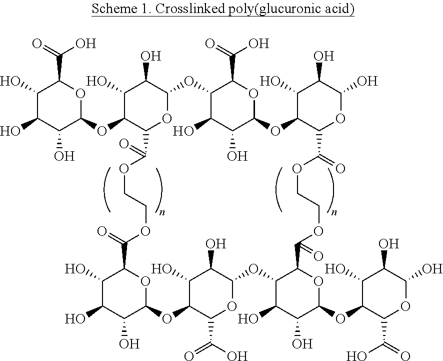

### Example 3

### Characterization of Hydrogen Bonding, Thermal and Mechanical Properties of Polysaccharide Hydrogels

Hydrogels made from mixtures of plant-based polysaccharides were studied. The intermediate strength hydrogel, Gel 1, was prepared from Konjac gum, Xanthan gum, κ-Carrageenan gum, and ι-Carrageenan gum as follows. Konjac gum (0.168 g), Xanthan gum (0.112 g), κ-Carrageenan (0.08 g), and ι-Carrageenan gum (0.16 g) were added to N-methyl pyrrolidone (1.2 g) and the mixture was stirred into water (17.09 g). Potassium chloride (0.08 g), calcium lactate (0.16 g), sodium benzoate (0.02 g), and COSMOCIL®CQ (0.02 g) were added and the mixture was stirred until the constituents were dissolved. Glucose (0.90 g) and malic acid (0.05 g) were mixed and then added to the solution and stirred in slowly until the solution thickened. The solution was weighed, heated to 85° C., and reweighed. The amount of water lost in the heating was re-added to the mixture and the mixture was again heated to 85° C. The gel was then poured into five separate Petri dishes (diameter 5 cm) in quantities of 2 g each and was allowed to cool at 4° C. to solidify. The resulting hydrogel was not dried.

A “strong” hydrogel was prepared using the method for preparing Gel 1 above, but omitting \acute{ι}-Carrageenan. The gel was dried for 3 days at 4° C. resulting in a weight loss of 82% (Table 4).

A “weak” hydrogel was prepared using the method for preparing Gel 1 above, but omitting xanthan gum. The resulting hydrogel was not dried.

The properties of each of these gels, as well as modified gels prepared according to the description above, are shown in Tables 3 and 4. These hydrogels or combinations thereof are the skin scaffolds in accordance with the invention. The results demonstrate that these skin scaffolds permit cells to be grown in them easily and function as dermis and epidermis layers depending on their Young's modulus.

TABLE 3
Thermal and Mechanical Properties of Polysaccharide Hydrogels

| | | | | | | | | | | | Weight | |
|---|---|---|---|---|---|---|---|---|---|---|---|---|
| | | | | | | | Modulus in | | Poisson's | | Loss from | |
| | HBV | | | | Tg | Mp | MPa | | Ratio | | Drying in | |
| Mixture | (day) | | TGA (° C.) | | (° C.) | (° C.) | (day) | | (day) | | % (day) | |
| Gel 1 | 690 | (d69) | 52 | (52%) | −32.2 | −14.19 | 0.02 | (d3) | | | | |
| | | | 183 | (14%) | | | 0.13 | (d6) | 0.04 | (d6) | 34% | (d6) |
| | | | | | | | 24.01 | (d8) | 0.07 | (d8) | 66% | (d8) |
| Gel 1 | 701 | (d38) | 95 | (63.5%) | −31.78 | −7.92 | Sample broke | | Sample | | Sample | |
| minus | | | 184 | (8.6%) | | | (d1, 2) | | broke | | broke | |
| Konjac | | | 215 | (2%) | | | 38.0 | (d3) | | | | |
| Gel 1 | 901 | (d1) | 183 | (34%) | NA | NA | 0.014 | (d1), | broke | (d1) | | |
| 150% Konjac | | | | | | | 0.019 | (d2), | 0.58 | (d2), | 53% | (d2), |
| | | | | | | | broke | (d3) | 4.92 | (d3) | 57% | (d3) |
| Gel 1 minus | 626 | (d17) | 47 | (32.5%) | −32.14 | Not | 0.015 | (d1), | Tore | (d1) | | |
| xanthan | | | 180 | (61%) | | seen | 0.019 | (d2), | 7.05 | (d2), | 49% | (d2), |
| | | | | | | | 0.020 | (d3) | 19.61 | (d3) | 93% | (d3) |

TABLE 4
Characterization of Hydrogels based on Plant-Based Polysaccharides

| | | | | | | | | | | | Weight | |
|---|---|---|---|---|---|---|---|---|---|---|---|---|
| | | | | | | | Modulus in | | Poisson's | | Loss from | |
| | HBV | | | | Tg | Mp | MPa | | Ratio | | Drying in | |
| Mixture | (day) | | TGA (° C.) | | (° C.) | (° C.) | (day) | | (day) | | % (day) | |
| Gel 1 | 690 | (d69) | 52 | (52%) | −32.2 | −14.19 | 0.02 | (d3) | | | | |
| | | | 183 | (14%) | | | 0.13 | (d6) | 0.04 | (d6) | 34% | (d6) |
| | | | | | | | 24.01 | (d8) | 0.07 | (d8) | 66% | (d8) |
| Gel 1 | 698 | (d33) | 69 | (74%) | −32.16 | Not | Broke (d1, 4) | | broke | | broke | |
| minus | | | 172 | (10%) | | seen | 0.019 | (d2) | | | | |
| κ-Carrageenan | | | | | | | | | | | | |
| Gel 1 | 691 | (d14) | 186 | | | Not | 0.024 | (d1), | | | | |
| 150% | | | (25.5%) | | | seen | 0.045 | (d3), | 0.30 | (d3) | 23% | (d3), |
| κ-Carrageenan | | | | | | | 21.75 | (d5) | 0.92 | (d5) | 83% | (d5) |
| Gel 1 | 727 | (d34) | 183 | (27%) | | Not | Broke | (d1) | | | | |
| minus KCl | | | | | | seen | 5.51 | (d2), | 0.91 | (d2), | 88% | (d2), |
| | | | | | | | 12.70 | (d3) | 0.63 | (d3) | 94% | (d3) |
| Gel 1 | 713 | (d29) | 69 | (73.5%) | −32.5 | Not | Broke | (d1), | | | | |
| minus | | | 179 | (8.5%) | | seen | 0.077 | (d2) | 7.87 | (d2), | 56% | (d2), |
| ι-Carrageenan | | | | | | | 56.21 | (d3) | 5.24 | (d3) | 82% | (d3) |
| Gel 1 minus | 716 | (d77) | 203 | (5%) | | | 6.99 | (d77) | | | | |
| Ca²⁺-Lactate | | | 224 | (7.5%) | | | | | | | | |

To determine the release properties of the hydrogels for various bioactive peptides, the degradation properties were determined. Degradation was found to begin within about 3 hours and continue for at least three weeks in weak acid (0.01M HCl). Such degradation may be correlated with the necessary release profile for different bioactive peptides.

It is to be understood that the invention is not limited in its application to the details of construction and the arrangement of components set forth in the preceding description or illustrated in the drawings. The invention is capable of other embodiments and of being practiced or of being carried out in various ways.

1-33. (canceled)

34. An artificial, biodegradable skin scaffold, comprising:
a biodegradable polysaccharide hydrogel composition having a Young's modulus of at least approximately 10 MPa.

35. The artificial, biodegradable skin scaffold according to claim 34, wherein said biodegradable polysaccharide hydrogel composition is a first biodegradable polysaccharide hydrogel composition and further comprising a second biodegradable polysaccharide composition, wherein said second biodegradable polysaccharide composition contacts said first biodegradable polysaccharide hydrogel composition.

36. The artificial, biodegradable skin scaffold according to claim 35, wherein said second biodegradable polysaccharide hydrogel composition has a Young's modulus of less than 10 MPa.

37. The artificial, biodegradable skin scaffold according to claim 34, wherein said biodegradable polysaccharide hydrogel composition comprises a polysaccharide selected from the group consisting of carrageenan, xanthan gum, locust bean gum, konjac gum, starch, methyl cellulose, carboxymethyl cellulose, ethyl cellulose, partially- or fully-deacetylated gellan, carob gum, agar, poly(glucuronic acid), poly(galacturonic acid) and a combination thereof.

38. The artificial, biodegradable skin scaffold according to claim 34, wherein said biodegradable polysaccharide hydrogel composition comprises a member selected from the group consisting of a glycol ether, a pyrrolidone, a carboxymethyl cellulose, an organic acid, a monosaccharide, a disaccharide, an antibacterial agent, a surfactant and a combination thereof.

39. The artificial biodegradable skin scaffold according to claim 34, wherein said biodegradable polysaccharide hydrogel composition comprises at least three polysaccharides selected from the group consisting of konjac gum, xanthan gum, K-carrageenan and I-carrageenan.

41. The artificial biodegradable skin scaffold according to claim 34, wherein said biodegradable polysaccharide hydrogel composition comprises a biological signal molecule.

42. The artificial biodegradable skin scaffold according to claim 41, further comprising a peptide, a protein or a pharmaceutical in combination with said biodegradable polysaccharide hydrogel composition.

43. The artificial biodegradable skin scaffold according to claim 34, further comprising a wound-healing peptide in combination with said biodegradable polysaccharide hydrogel composition.

44. The artificial biodegradable skin scaffold according to claim 34, further comprising skin cells in combination with said biodegradable polysaccharide hydrogel composition.

45. The artificial biodegradable skin scaffold according to claim 44, wherein said skin cells are autologous.

46. The artificial biodegradable skin scaffold according to claim 44, wherein said skin cells are selected from the group consisting of fibroblasts, keratinocytes and a combination thereof.

47. An artificial, biodegradable skin scaffold, comprising:
a biodegradable polysaccharide hydrogel composition comprising a biological signal molecule.

48. The artificial, biodegradable skin scaffold according to claim 47, wherein said biodegradable polysaccharide hydrogel composition has a Young's modulus of less than about 10 MPa.

49. The artificial biodegradable skin scaffold according to claim 47, wherein said biological signal molecule comprises a peptide, a protein or a pharmaceutical in combination with said biodegradable polysaccharide hydrogel composition.

50. A method for preparing an artificial, biodegradable skin scaffold, comprising the steps of:
preparing a biodegradable polysaccharide hydrogel composition; and,
drying said biodegradable polysaccharide hydrogel composition,
wherein said biodegradable polysaccharide hydrogel composition has a Young's modulus of at least approximately 10 MPa after drying.

51. The method for preparing an artificial, biodegradable skin scaffold according to claim 50, further comprising the steps of:
preparing an additional biodegradable polysaccharide hydrogel composition; and,
drying said additional biodegradable polysaccharide hydrogel composition,
wherein said additional biodegradable polysaccharide hydrogel composition has a lower Young's modulus than said biodegradable polysaccharide hydrogel composition.

52. The method for preparing an artificial, biodegradable skin scaffold according to claim 51, further comprising the step of:
seeding said additional biodegradable polysaccharide hydrogel composition with keratinocytes.

53. The method for preparing an artificial, biodegradable skin scaffold according to claim 51, wherein said biodegradable polysaccharide hydrogel composition and said additional biodegradable polysaccharide composition each comprise a member selected from poly(glucuronic acid), poly(galacturonic acid) and a combination thereof.
